(19) United States
(12) Patent Application Publication
(10) Pub. No.: US 2023/0050906 A1
(43) Pub. Date: Feb. 16, 2023

(54) HIGH-SURFACE AREA ELECTRODES FOR WEARABLE ELECTROCHEMICAL BIOSENSING

(71) Applicants: Amanda H. Imamura, Butantã, São Paulo (BR); THE REGENTS OF THE UNIVERSITY OF CALIFORNIA, Oakland, CA (US)

(72) Inventors: Julia A. Zakashansky, Irvine, CA (US); Michelle Khine, Irvine, CA (US); Amanda H. Imamura, Butantã (BR)

(21) Appl. No.: 17/790,659

(22) PCT Filed: Jan. 7, 2021
(86) PCT No.: PCT/US21/12503

§ 371 Date: Jul. 1, 2022

Related U.S. Application Data
(60) Provisional application No. 62/991,956, filed on Mar. 19, 2020.
(60) Provisional application No. 62/958,092, filed on Jan. 7, 2020.

(51) Int. Cl. G01N 27/327; B32B 15/08; B32B 37/00; B32B 3/30; B32B 27/28; A61B 5/1477; A61B 5/145

(52) U.S. Cl. CPC G01N 27/327; B32B 15/08; B32B 37/025; B32B 3/30; B32B 27/283; A61B 5/1477; A61B 5/14532; A61B 5/14546; B32B 2255/06; B32B 2255/205; B32B 2457/14; B32B 2535/00; B32B 2383/00; B32B 2311/04; A61B 2562/125

(57) ABSTRACT

The present invention is directed to the production of stretchable wrinkled film electrodes for use in wearable/portable ROC systems using electrochemical analysis techniques. A polymer layer is disposed on a conductive substrate and a sacrificial layer is disposed on said polymer layer. An electrode shape template is cut out of adhesive and disposed on the sacrificial layer. A metallic film is disposed on the sacrificial layer by the electrode shape template. The disposed layers are removed from the conductive substrate and placed in an oven to allow said layers to shrink. The shrunken metallic film is treated with a solution to promote bonding between the film and an elastomer. The elastomer is drop-cast onto the shrunken film and the sacrificial layer is dissolved to detach the shrunken polymer layer. The shrunken film and elastomer are placed in a chemical bath and dried, producing the stretchable wrinkled film electrode.

### CROSS-REFERENCES TO RELATED APPLICATIONS

This application claims benefit of U.S. Provisional Application No. 62/958,092 filed Jan. 7, 2020, and U.S. Provisional Application No. 62/991,956 filed Mar. 19, 2020, the specification(s) of which is/are incorporated herein in their entirety by reference.

### FIELD OF THE INVENTION

The present invention is directed to the fabrication and use of stretchable wrinkled electrodes for electrochemical detection of biomarkers in body fluids, more particularly to a device to be used in portable/wearable point-of-care health monitoring devices.

### BACKGROUND OF THE INVENTION

It is frequently possible to delay the onset or progression of an illness by identifying the cause and acting against it. Unfortunately, access to early detection is an issue; hospital visits and laboratory tests are expensive. Wearable sensors and point-of-care (POC) devices bring personalized healthcare to more people at a lower cost. Detailed insight into the user's health state can be obtained at the molecular level with analysis of biomarkers present in sweat, such as glucose, ethanol, lactic acid, ions (Na+, K+, Cl), and cortisol.

For example, cortisol is a widely accepted biomarker for physiological and emotional stress and exists in two forms in human bodies: bound to another molecule, or not bound (free). Monitoring fluctuations in the concentration of free cortisol can help identify and reduce harmful responses to stress, but invasive sample collection confounds data because of the difficulty of distinguishing the origin of the stress response. Through the non-invasive collection of saliva, the inherent stress response to blood collection is avoided. Electrochemical detection of these biomarkers is attractive because it can achieve clinically relevant sensitivity and accuracy and is easily integrated into electronic systems.

Biosensors that detect analytes in sweat face the challenge of maintaining sensitivity upon miniaturization. Various materials and processes have been developed to create nanostructured electrodes with high surface areas to mitigate this issue. The need remains, however, for biocompatible materials that can be scalably integrated into wearable devices. Integrating electrochemical transducers into wearable devices necessitates miniaturization of the electrodes. This negatively impacts the signal-to-noise ratio of the electrical response, as current is directly related to the sensing electrode surface area. To overcome this issue, nanostructured electrodes with small geometric footprints have been fabricated to achieve a higher surface area than their planar counterparts. Wearable sensors in particular should be biocompatible and withstand physiological strains due to motion and deformation of human skin, which is approximately 30%.

Stretchable gold electrodes have been evaluated as glucose detection platforms. It was previously demonstrated that the stability at strains up to 230% with a solution-processed wrinkled gold glucose sensor, with a limit of detection (LOD) of 1×10⁻³ M in artificial sweat. Additionally, it was reported that gold nanowire-based electrodes functionalized with glucose oxidase that were stretched to 20% strain to detect glucose concentrations of 1×10⁻⁵ M in a solution of NaOH, while it was demonstrated that a gold-fiber based sensor functionalized with glucose oxidase capable of detecting 6×10⁻⁶ M glucose in phosphate-buffered solution (PBS) under strains up to 200%. It is critical to have low detection limits and high sensitivity due to a lower concentration of glucose in sweat in comparison with other biological fluids. In this work, a highly stretchable electrode is introduced with the lowest limit of detection to the best of our knowledge for flexible enzyme-free glucose sensing at physiological pH.

### BRIEF SUMMARY OF THE INVENTION

It is an objective of the present invention to provide electrodes and fabrication methods for said electrodes that allow for electrochemical, impedimetric, capacitive, and colorimetric detection of biomarkers in body fluids, as specified in the independent claims. Embodiments of the invention are given in the dependent claims. Embodiments of the present invention can be freely combined with each other if they are not mutually exclusive.

In some aspects, a gold thin-film electrode is fabricated using a thermoplastic shape memory polymer to create hierarchical wrinkled structures via a miniaturization process, followed by transfer onto a soft, wearable substrate. In some embodiments, the original electrochemically active surface area (EASA) of the unshrunk electrode is retained in the final flexible electrode, thereby allowing for more reaction sites than on a planar electrode of the same geometric area.

In some embodiments, the flexible electrode sensitively detects glucose without enzymes or additional labels at physiological pH in the range of 1×10⁻⁷-1×10⁻⁴_{M} with a calculated limit of detection (LOD) of 2.22×108⁻⁸_{M}, the lowest ever reported for an enzyme-free sensor on a stretchable substrate. This concentration range is relevant for glucose detection in saliva, sweat, or tears. Diabetic patients seeking to monitor their blood glucose can do so without having to draw blood, and without enzymes, the sensor can have a longer shelf life and be commercialized for in-home use.

The preliminary data for glucose detection indicate that other biomarkers can similarly be detected in very low concentrations, which is important for detecting relevant concentration changes in body fluids in response to stress stimuli, disease progression, and response to medication, among other diagnostic applications.

Although biosensors detect analytes portably and noninvasively, they face the challenge of maintaining sensitivity upon miniaturization. Various materials and processes have been developed to create nanostructured electrodes with high surface areas to mitigate this issue; however, the need remains for biocompatible materials that can be integrated into wearable or point-of-care (POC) devices.

The present invention features methods for a stretchable wrinkled electrode and the fabrication and use of the electrode for electrochemical detection of biomarkers in body fluids, to be used in portable/wearable point-of-care health monitoring devices.

One of the unique and inventive technical features of the present invention is the transfer of a shrunken wrinkled metallic film to a flexible substrate through the use of an oxygen plasma treatment. Without wishing to limit the invention to any theory or mechanism, it is believed that the technical feature of the present invention advantageously provides for even additional electroactive surface area (EASA) of the electrode, allowing for improved signal detection. None of the presently known prior references or work has the unique inventive technical feature of the present invention.

Furthermore, the prior references teach away from the present invention. For example, prior systems teach the transfer of a wrinkled electrode to a substrate through the use of a super wetting polymer in place of an oxygen plasma treatment of the metallic film. Thus, the prior art teaches away from the inventive feature of the present invention. Surprisingly, the oxygen plasma treatment provides better surface wetting while retaining almost all of the electrochemically active surface area of the non-shrunken metallic film.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows an outline of a method for fabricating stretchable wrinkled electrodes for electrochemical sensing.

FIG. 2A shows a photo of gold electrodes, from left to right: unshrunk electrode, a shrunk electrode on a polyolefin layer, and a shrunk electrode on an elastomer substrate.

FIGS. 2B-2D show a scanning electron microscope (SEM) photo of an unstretched, transferred electrode at 1.1 (FIG. 2C) and 12.9 k× magnification (FIG. 2D).

FIGS. 2E-2F shows a photo of a transferred electrode being stretched. FIGS. 2E-2F shows a scanning electron microscope (SEM) of the stretched transferred electrode at 1.1 (FIG. 2E) and 12.9 k× (FIG. 2G) magnification.

FIGS. 3A-3B show a graph of the normalized change in resistance across 100 strain cycles (FIG. 3A) and 10 (FIG. 3B) strain cycles. The electrode was strained to 200% of the original length. This demonstrates the mechanical stability at hundreds of cycles during which the electrode was strained to 210% of its original length.

FIGS. 4A-4B show current density voltammograms of unshrunk (triangle), shrunk (square), and transferred (circle) electrodes in H₂SO₄ (FIG. 4A) and in [Fe(CN)₆]_{3-/4-} (FIG. 4B).

FIG. 4C shows a graph of shrinking factors and signal enhancements for shrunk (square) and transferred (circle) gold electrodes, expressed as ratios.

FIGS. 4D-4E show cyclic voltammograms of transferred gold electrodes stretched up to 0 to 210% of its length in H₂SO₄ (FIG. 4D) and in [Fe(CN)_{6]3-/4-} (FIG. 4E).

FIG. 4F shows a graph of the signal increase of transferred gold electrodes as measured by peak height before and after stretching up to 210% of its length and gain in signal in H₂SO₄ and [Fe(CN)_{6]3-/4-}.

FIGS. 4G-4H show cyclic voltammograms of transferred gold electrodes in H₂SO₄ (FIG. 4G) and [Fe(CN)_{6]3-/4-} (FIG. 4H) before and after stretching to 210% of its length and relaxing overnight.

FIG. 4I shows a graph of the signal increase of transferred gold electrodes after relaxing overnight as measured by peak height before and after stretching up to 210% of its length in H₂SO₄ and [Fe(CN)_{6]3-/4-}. The signal is higher after stretching and also has a higher signal to noise ratio.

FIG. 5A and FIG. 5C shows amperometry detection of glucose from 1×10⁻⁶M-1×10⁻³M in PBS using unstrained electrodes (FIG. 5A) and pre-strained electrodes (FIG. 5C).

FIG. 5B and FIG. 5D shows the linear correlation of current values with glucose concentration in unstrained electrodes (R₂=0.99, FIG. 5B) and in pre-strained electrodes (R₂=0.99, FIG. 5D).

FIGS. 6A-6B show cyclic voltammograms of unshrunk, shrunk, and transferred Au electrodes in H₂SO₄ (FIG. 6A) and [Fe(CN)_{6]3-,4-} (FIG. 6B) at 1000 mV s⁻¹.

FIG. 6C shows a table of charge and surface area of shrunk and transferred Au electrodes.

FIGS. 7A-7B show the power density as a function of scan rate for unshrunk, shrunk, and transferred electrodes in H₂SO₄ (FIG. 7A) and in [Fe(CN)_{6]3-,4-} (FIG. 7B)

FIG. 7C shows the normalized ratio of increase in signal enhancement (ratio at a given scan rate divided by ratio calculated at lowest scan rate, 10 mV s⁻¹) as a function of increasing scan rate.

FIG. 8A shows a diagram of crack formation on the transferred gold electrode with strain.

FIG. 8B shows a table of sheet resistance of transferred Au electrodes before straining, during strain at 200%, and after relaxation.

FIG. 9A-9B show a cyclic voltammogram of wrinkled electrodes on Ecoflex in a phosphate buffer and 5×10⁻³ M glucose (denoted by an arrow). FIG. 9B is zoomed in on the oxidation peak (dashed box in FIG. 9A).

FIG. 10 shows a schematic of different states of fabricating and transferring a stretchable wrinkled electrode.

FIG. 11 shows the process flow to create SDW E-AB sensors. First, a thin layer of gold is sputtered onto polystyrene plastic, which is shrunk to create wrinkles (SEM inset shows representative wrinkle morphology). The wrinkled surface is incubated with aptamers conjugated with methylene blue (MB), then incubated with 6-mercaptohexanol (MCH) as the blocking molecule. After functionalization, the wrinkled surface was exposed to the 51 protein. Arrows indicate change in electron transfer with and without the spike protein attached (through the RNA binding domain (RBD)). Graph illustrates change in current due to the change in electron transfer for spike bound MB on SDW electrodes upon addition of 51 protein.

FIG. 12A shows the beaker cell configuration with CD electrode.

FIG. 12B shows the raw peak height change in current with increasing concentrations of 51 protein in phosphate buffer solution on CD electrodes. Peaks decrease in height as the concentration of 51 increases on the graph.

FIG. 12C shows the titration curves collected at various frequencies.

FIG. 13A shows the beaker cell configuration with SWD electrode.

FIG. 13B shows the raw peak height change in current with increasing concentrations of S1 protein in 10% saliva on SDW electrodes.

FIG. 13C shows the normalized change in signal produced from sequential incubations of saliva spiked with increasing concentrations of S1 protein minus signal from sequentially incubated blank saliva samples. Peaks decrease in height as the concentration of S1 increases on the graph. Hill fit represented denoted by an arrow.

FIG. 13D shows the methylene blue absolute peak current and peak current density comparison between CD and SDW electrodes with equivalent geometric areas.

FIG. 14A shows a comparison of change in area under the gold reduction peak during cycling in sulfuric acid between individual SDW electrodes and SDW mini cells. Peak height and area under the peak stabilized by 120 cycles at 1000 mV s-1.

FIG. 14 B shows the Sensit Smart (Palmsens) potentiostat next to a SDW mini cell with scale provided in centimeters. Electrode arrangement (left to right): counter (60 nm Au), working (60 nm Au) and reference (Ag/AgCl ink).

FIG. 14C shows the titration curve of S1 protein performed at 10 Hz in 10% saliva. Signal change normalized to 10% saliva without S1 protein.

### DETAILED DESCRIPTION OF THE INVENTION

Following is a list of elements corresponding to a particular element referred to herein:
10 conductive substrate
20 polymer layer
30 sacrificial layer
40 metallic film
60 electrode shape template
120 shrunken polymer layer
130 shrunken sacrificial layer
140 shrunken metallic film
150 elastomer
200 stretchable wrinkled electrode

The electrode of the present invention is stretchable and unobtrusive, making it an excellent candidate for wearable health monitoring. The geometric area of the flexible electrode is approximately 30 times smaller than that of the unshrunk electrode. Remarkably, a further enhancement to signal is achieved upon stretching the electrode to 210% of its original length then relaxing it overnight. Stretching the electrode also aids diffusion limited reactions by “unraveling” some of the wrinkles, allowing diffusion to all of the electrochemically active surface area (EASA) and producing a higher signal than its unstretched counterpart.

The electrode has the lowest limit of detection (LOD) of glucose of any reported non-enzymatic, stretchable sensor thus far, which is crucial to detecting small changes in low concentrations of glucose. This suggests that similarly low detection limits can be achieved for other biomarkers, like ethanol, lactic acid, ions (Na+, K+, Cl—), and cortisol. Additionally, the lack of enzymes ensures no degradation of sensor components and promotes scalable manufacturing for commercialization.

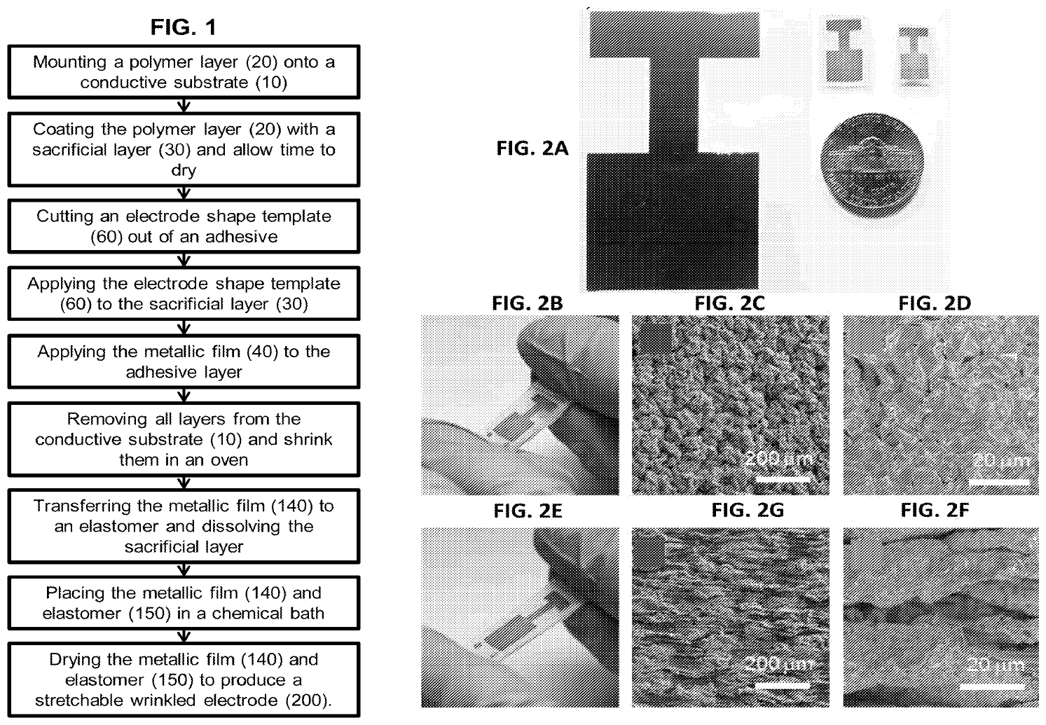

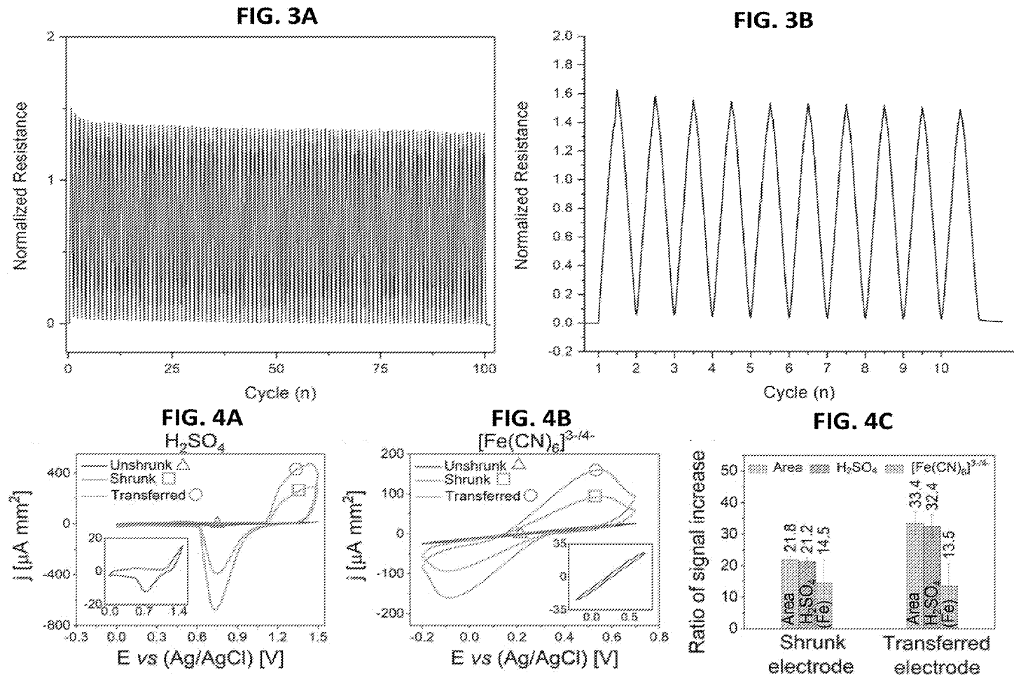

Referring now to FIGS. 1-140, the present invention features methods for the fabrication and use of a stretchable wrinkled electrode for electrochemical detection of biomarkers in body fluids, to be used in portable/wearable point-of-care health monitoring devices.

In some embodiments, body fluids may refer to but are not limited to, saliva, sweat, tears, Cerebrospinal fluid, serum, or urine.

As used herein “biomarkers” or “analytes” may be used interchangeably and may refer to a substance whose chemical constituents are being identified and measured. In some embodiments, the measured amount of a substance may be indicative of some phenomenon such as disease, infection, or environmental exposure.

In some embodiments, non-limiting examples of biomarkers may include but are not limited to, glucose, ethanol, lactic acid, cortisol, or ions such as but not limited to sodium, potassium, or chloride.

In some embodiments, non-limiting examples of analytes may include but are not limited to cortisol or viral proteins.

The present invention may feature a method for producing stretchable wrinkled electrodes (200) for electrochemical, impedimetric, capacitive, and colorimetric sensing. In some embodiments, the method may comprise mounting a polymer layer (20) onto a conductive substrate (10). The polymer layer (20) may then be coated with a sacrificial layer (30). After allowing the sacrificial layer (30) to dry, an electrode shape template (60) may be cut out of an adhesive and applied on top of the sacrificial layer (30). The electrode shape template (60) may act as a stencil for the deposition of a metallic film (40). In some embodiments, the method may further comprise depositing the metallic film (40) on top of the sacrificial layer (30) by the electrode shape template (60). After depositing the metallic film (40), the electrode shape tem on the sacrificial layer (30). The polymer layer (20), sacrificial layer (30), and metallic film (40) may then be removed from the conductive substrate (10) and placed in an oven to create a shrunken polymer layer (120), a shrunken sacrificial layer (130) and a shrunken metallic film (140). In further embodiments, the method comprises treating the shrunken metallic film (140) with a first solution. In some embodiments, treating the shrunken metallic film (140) promotes bonding between the shrunken metallic film (140) and an elastomer (150). The elastomer (150) may then be drop-casted onto the shrunken metallic film (140) such that the shrunken metallic film (140) bonds to the elastomer (150). The shrunken sacrificial layer (130) between the shrunken metallic film (140) and the shrunken polymer layer (120) may be dissolved using a second solution, such that the shrunken metallic film (140) detaches from the shrunken polymer layer (120). In some embodiments, the method comprises placing the shrunken metallic film (140) and the elastomer (150) in a chemical bath and drying the shrunken metallic film (140) and the elastomer (150) to produce the stretchable wrinkled electrode (200).

The present invention may also feature a method for producing stretchable wrinkled electrodes (200) for electrochemical, impedimetric, capacitive, and colorimetric sensing. In some embodiments, the method may comprise mounting a polymer layer (20) onto a conductive substrate (10). The polymer layer (20) may then be coated with a sacrificial layer (30). In some embodiments, the method comprises applying the electrode shape template (60) on top of the sacrificial layer (30) and depositing a metallic film (40) on top of the sacrificial layer (30) by the electrode shape template (60). In further embodiments, the method comprises removing the polymer layer (20), sacrificial layer (30), and metallic film (40) from the conductive substrate (10). In some embodiments, the method comprises shrinking the polymer layer (20), sacrificial layer (30), and metallic film (40) to create a shrunken polymer layer (120), a shrunken sacrificial layer (130), and a shrunken metallic film (140), treating the shrunken metallic film (140) with a first solution, wherein the first solution (140) promotes bonding between the shrunken metallic film (140) and an elastomer (150), and dissolving the shrunken sacrificial layer (130) between the shrunken metallic film (140) and the shrunken polymer layer (120) using a second solution. In some embodiments, the method further comprises producing the stretchable wrinkled electrode (200) comprising a shrunken metallic film (140) and the elastomer (150).

In some embodiments, the polymer layer (20) or the shrunken polymer layer (120) may comprise polyolefin (PO). In some embodiments the polymer layer (20) or the shrunken polymer layer (120) may comprise polystyrene. In some embodiments the polymer layer (20) or the shrunken polymer layer (120) may comprise any thermoplastic shape-memory polymer.

In some embodiments, the conductive substrate (10) may comprise a silicon wafer. In some embodiments, the conductive substrate (10) is glass. In other embodiments, the conductive substrate (10) is not conductive. In some embodiments, the conductive substrate (10) is any rigid support.

In some embodiments, the sacrificial layer (30) or the shrunken sacrificial layer (130) may comprise poly(methyl methacrylate) (PMMA) dissolved in toluene. In other embodiments, the sacrificial layer (30) is applied to the polymer layer (20) via spin coating. In some embodiment, an adhesive may comprise frisket film.

In some embodiments, the metallic film (40) or the shrunken metallic film (140) may comprise gold. In some embodiments, the metallic film (40) or the shrunken metallic film (140) may comprise platinum. In some embodiments, the metallic film (40) or the shrunken metallic film (140) may comprise silver. In some embodiments, the metallic film (40) or the shrunken metallic film (140) may comprise copper. In some embodiments, the metallic film (40) or the shrunken metallic film (140) may comprise carbon. In some embodiments, the metallic film (40) or the shrunken metallic film (140) may comprise any conductive material. In other embodiments, the metallic film may be deposited onto the sacrificial layer (30) via sputtering.

In some embodiments, the electrode (200) is placed in a convection oven. In other embodiments, the electrode (200) is placed in a conventional oven. In preferred embodiments, the electrode (200) is placed in an oven at 140° C. In other embodiments, the electrode (200) is placed in an oven ranging in temperature from about 125° C. to 145° C. In preferred embodiments, the electrode (200) is placed in an oven for 13 minutes. In other embodiments, the electrode (200) is placed in an oven for about 10 to 15 minutes.

In some embodiments, the first solution comprises silane. In other embodiments, the first solution promotes bonding. In other embodiments the second solution comprises acetone. In further embodiments, the second solution may cause the shrunken metallic film (140) detaches from the shrunken polymer layer (120). In some embodiments, the chemical bath comprises isopropanol. In other embodiments, the chemical bath comprises toluene.

As used herein, “elastomer” may refer to a polymer with viscoelasticity and has very weak intermolecular forces, generally low Young's modulus and high failure strain compared with other materials. In some embodiments, the elastomer (150) is silicon-based. In some embodiments, the elastomer (150) is any material which fits the aforementioned definition of an elastomer.

In some embodiments, the stretchable wrinkled electrode (200) has an average geometrical area of 0.2 cm² to 0.4 cm². In some embodiments, the electrode (200) has an area of about 0.1 cm² to 0.15 cm². In some embodiments, the electrode (200) has an area of about 0.2 cm² to 0.25 cm². In some embodiments, the electrode (200) has an area of about 0.3 cm² to 0.35 cm². In some embodiments, the electrode (200) has an area of about 0.4 cm² to 0.45 cm². In some embodiments, the electrode (200) has an area of about 0.5 cm² to 0.55 cm². In some embodiments, the electrode (200) has an area of 0.6 cm². In some embodiments, the electrode (200) has an area of about 0.1 cm², or about 0.15 cm², or about 0.2 cm², or about 0.25 cm², or about 0.3 cm², or about 0.35 cm², or about 0.4 cm², or about 0.45 cm², or about 0.5 cm², or about 0.55 cm², or about 6.0 cm². In other embodiments, the electrode has an area greater than 6.0 cm². In some embodiments, the final size of the stretchable wrinkled electrode (200) depends on the initial working area of the electrode (i.e., before the electrode is shrunk). In other embodiments, the amount the initial electrode (i.e., before the electrode is shrunk) shrinks depends on the type of polymer and/or the amount of time spent shrinking.

In some embodiments, the stretchable wrinkled electrode comprises hierarchical, wrinkled structures. In some embodiments, a “hierarchical wrinkled structure” may refer to a larger wrinkle on the electrode having wrinkles (FIGS. 2C-2D). In some embodiments, depending on the amount the polymer shrinks there may be more wrinkles. In some embodiments, the first wrinkles to form are very small, as the polymer begins shrinking and the gold (or other suitable materials) begins to accommodate the smaller substrate size. As the shrinking continues, the gold continues to wrinkle and buckle, creating bigger-sized wrinkles

Referring to FIG. 10, the present invention features a stretchable wrinkled electrode (200) for electrochemical, impedimetric, capacitive, and colorimetric sensing. In some embodiments, the surface of the stretchable wrinkled electrode (200) can be modified for selective sensing of specific analytes using electrochemical, impedimetric, capacitive, and colorimetric sensing. The electrode (200) may comprise an elastomer (150) and a shrunken metallic film (140). In some embodiments, the shrunken metallic film (140) is fabricated by depositing a metallic film (40) on top of a polymer layer (20) by a sacrificial layer (30), placing the polymer layer (20), the sacrificial layer (30), and the metallic film (40) in an oven such that a shrunken polymer layer (120), a shrunken sacrificial layer (130), and the shrunken metallic film (140) are created, and dissolving the shrunken sacrificial layer (130) to detach the shrunken polymer layer (120). In other embodiments, the shrunken metallic film (140) may have been treated with a first solution, such that the first solution promotes bonding between the shrunken metallic film (140) and the elastomer (150). In further embodiments, the shrunken metallic film (140) may attach to the elastomer (150) and the electrode (200) may be placed in a chemical bath and dried after construction and prior to use.

In some embodiments, the elastomer (150) may be silicon-based. In some embodiments, the metallic film (40) and the shrunken metallic film (140) may comprise gold. Non-limiting examples of materials that the metallic film (40) or the shrunken metallic film (140) may comprise may include but are not limited to platinum, silver, copper, carbon, or any other conductive material.

In some embodiments, the polymer layer (20) and the shrunken polymer layer (120) may comprise polyolefin. In some embodiments the polymer layer (20) or the shrunken polymer layer (120) may comprise polystyrene. In some embodiments the polymer layer (20) or the shrunken polymer layer (120) may comprise any thermoplastic shape-memory polymer.

In some embodiments, the sacrificial layer (30) and the shrunken sacrificial layer (130) may comprise poly(methyl methacrylate) (PMMA) dissolved in toluene. In other embodiments, the shrunken sacrificial layer (130) is dissolved using a second solution comprising acetone

In some embodiments, the first solution may comprise a silane. In other embodiments, the first solution promotes bonding. In other embodiments, the second solution may comprise acetone. In some embodiments, the chemical bath may comprise isopropanol or toluene.

An exemplary embodiment of the present invention features a method for fabricating a wrinkled electrode, as follows. PO is mounted onto a silicon wafer to be coated with poly(methyl methacrylate) (PMMA) (Sigma Aldrich) dissolved in toluene (22.5 mg mL-1) to comprise the sacrificial layer. The surface tension of the solvent in which the PMMA is dissolved is taken into consideration to achieve good wetting with the surface energy of the PO, which is in the range of 30 dynes cm². The surface energy for polyolefins is relatively low, and favors wetting by the lower-surface energy solution. Toluene (28.40 dyne cm⁻¹ at 20° C.) has a lower surface tension than the previously used solvent, anisole (35.00 dyne cm⁻¹ at 20° C.). The PMMA is spin coated at 3200 rpm to ensure uniform spreading and reduce the thickness of the sacrificial layer and subsequently the time necessary to dissolve it. After drying the PMMA film overnight, masks for the electrode design are laser-cut from adhesive and applied to the films as stencils for gold deposition. The initial working area of the electrode was 10.5 cm². An 80 nm gold film is deposited on top of the PMMA-coated PO via sputtering (Quorum 150R). The PO-PMMA-Au is then removed from the silicon wafer substrate and prepared for thermal treatment. The films are shrunk in a convection oven at 140° C. for 13 min. The average geometrical area of shrunk electrode is 0.48 cm².

An exemplary embodiment of the present invention features a method for transferring a wrinkled electrode to an elastomer, as follows. The wrinkled gold film is treated with a silane solution to promote bonding to the flexible silicon-based elastomer prior to the liftoff procedure. The elastomer is drop casted onto the silane-treated gold film and allowed to cure overnight at 60° C. The wrinkled films are transferred to the Ecoflex by dissolving the PMMA between the gold and polyolefin in acetone. Once the films are fully transferred, they are placed in an isopropanol bath for an hour and dried overnight. The average geometrical area of the transferred, working electrode is 0.31 cm². SEM images of samples prepared with sputtered iridium (4 nm) are taken using the GAIA Tescan system (5 kV, 0.20 pA). The average geometric area of the shrunk and transferred electrodes is 0.48 and 0.31 cm², respectively. Samples are prepared for SEM imaging with sputtered iridium (6 nm) and are taken using the GAIA Tescan system (8 kV, 0.20 pA).

In some embodiments, overnight may refer to a timeframe between 4 and 18 hours.

An exemplary embodiment of the present invention is tested using electrochemical characterization, as follows. Electrochemical characterization is performed on a Reference 600 potentiostat (Gamry Instruments) with the unshrunk, shrunk and transferred electrodes as working electrodes, platinum (Pt) wire as the counter electrode and silver/silver chloride (Ag/AgCl) as the reference electrode. Cyclic voltammetry is firstly performed in a H₂SO₄ 0.5 _{M} solution from 0 to 1.5 V at 10, 25, 50, 100, 250, 500 and 1000 mV s-1. The process is repeated in 0.005 _{M} [Fe(CN)_{6]3-/4-} solution in 0.05 M PBS solution at pH 7.4 from −0.2 to 0.7 V.

An exemplary embodiment of the present invention is tested using electrochemical characterization on transferred electrodes under strain, as follows. A stretching system was developed to maintain the flexible electrodes under strain while an electrochemical measurement was performed. Cyclic voltammetry is performed at every 30% strain of electrode length in H₂SO₄ and [Fe(CN)_{6]3-/4-} solutions until it reaches 210% of its original length. The electrode is allowed to relax overnight before repeating the stretching procedure.

An exemplary embodiment of the present invention is tested by calculating surface area by charge associated with gold oxide reductions, as follows. The electroactive surface area (EASA) of the nanostructured electrode is evaluated by gas sorption and by several electrochemical measurements, such as underpotential deposition of metal, capacitance measurement by electrochemical impedance spectroscopy (EIS) or cyclic voltammetry, and, most commonly used cyclic voltammetry in H₂SO₄. The latter technique is widely applied to characterize nanostructured gold surfaces because it can be easily performed and provides a good estimate of the surface area. The surface area estimated by gold oxide reduction was reported to agree with the areas measured by underpotential deposition (UPD) and nitrogen adsorption/desorption isotherms. In fact, it was previously demonstrated that the gold oxide reduction in H₂SO₄ was proven as the most accurate and reliable method to determine the real area when compared to several other methods such as assessment of monolayer (copper, 6(ferrocenyl)hexanethiol and cytochrome c), redox reactions ([Fe(CN)_{6]3-/4-}, [Ru(NH₃)_{6]3-/4-} and O₂) and double layer capacitance. The measurement of gold electrode by cyclic voltammetry in H₂SO₄ consists in the formation and successive reduction of gold oxide and the charge associated with this reaction at a relatively slow scan rate is directly proportional to the surface area by the following relation:

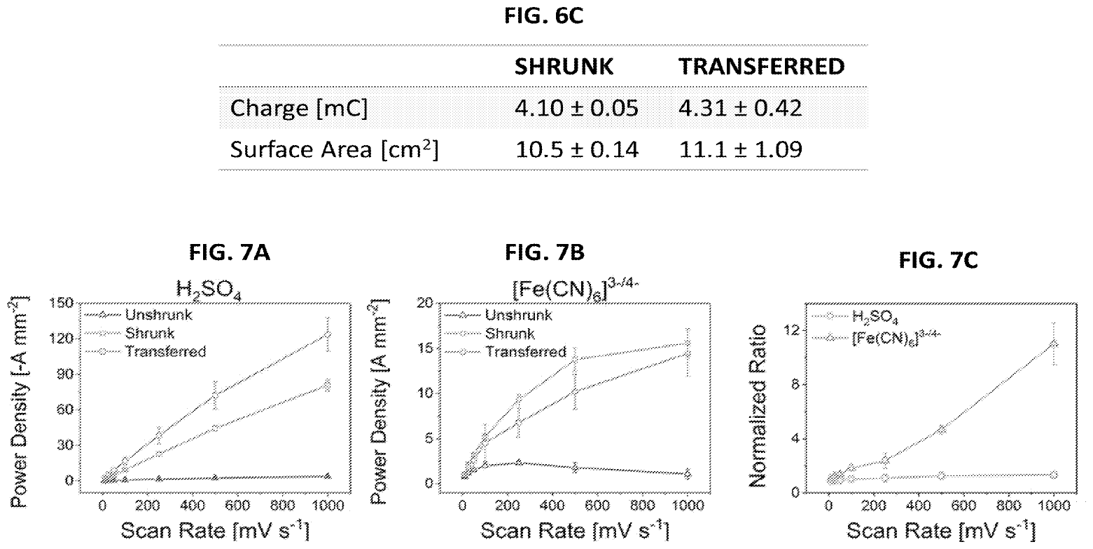

EASA=Q/c

where Q is charge which is calculated by integrating the area under the reduction peak and c is the specific charge equivalent, which equals to 390 ρC cm⁻² for polycrystalline gold. FIG. 6C shows the calculated values for the electroactive surface area of shrunk and transferred Au electrodes based on the integrated area under the reduction peak at 100 mV s⁻¹. Despite the limitations of this method to accurately assess surface area due to the possibility of multilayer formation instead of monolayer, this method provides an approximated value of shrunk and transferred surfaces areas with the initial geometric area of the unshrunk electrode. It is concluded that the surface area is maintained after shrinking and transferring the thin film when the reduction peak of gold oxide is evaluated

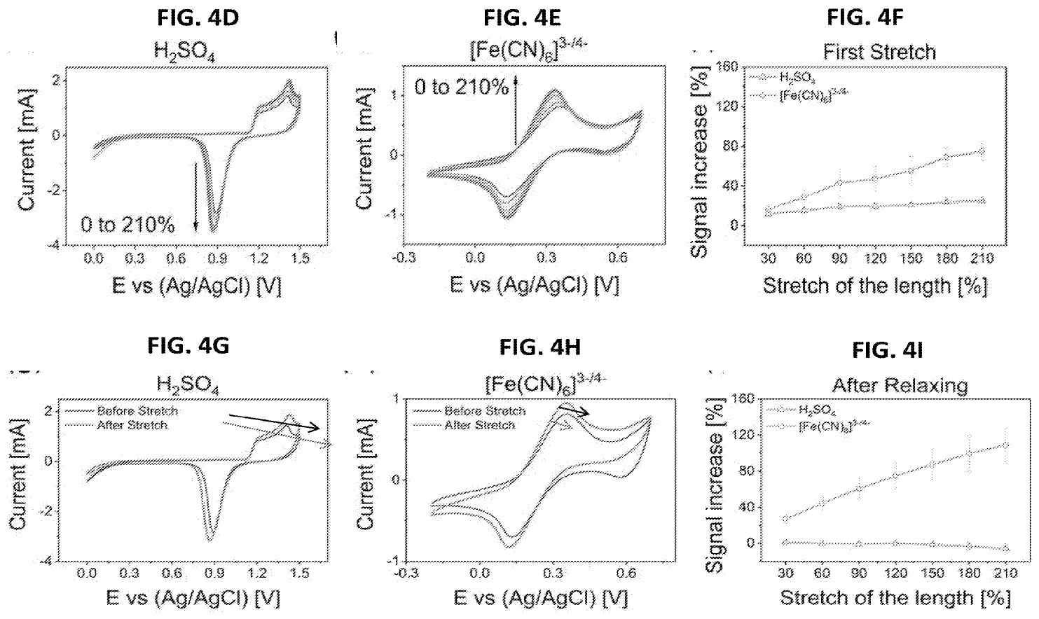

An exemplary embodiment of the present invention is tested by calculating the dependency of signal enhancement with scan rate, as follows. Diffusion-limited redox reactions, such as for the redox par [Fe(CN)_{6]-3/-4}, is affected by the scan rate of the cyclic voltammetry. At fast scan rates, the redox species are forced to move more rapidly, which results in the decrease of the diffusion layer. Consequently, higher currents are observed since more redox species are able to access the surface area. The dependency of the Faradaic current from diffusion-limited reactions on scan rate is described by the Randles-Sevcik equation:

$${ι}_{ρ = 0.446 {{nFAC}^{0} ( \frac{{nFvD}_{0}}{RT} )}^{1 / 2}}$$

where n is the number of electrons involved in the electrochemical reaction, F is the Faraday constant, C₀ is the concentration of the redox species, v is the scan rate, D₀ is the diffusion coefficient of the oxidized analyte, R is the gas constant, and T is the temperature. As shown in FIG. 4B and FIG. 6B, at high scan rates the shape of the voltammogram for the unshrunk electrode is deformed from the well-known “duck”-shaped voltammogram of [Fe(CN)_{6]3-/4-}, in which it is hard to define the oxidation and reduction peaks. Although, the peaks are well defined for shrunk and transferred electrodes, which can be explained by some of the [Fe(CN)_{6]3-/4-} species trapped in the wrinkles, preventing them to move away from the electrode surface. Therefore, power values, area under the voltammogram, were calculated to evaluate the dependency with scan rate, as shown in FIGS. 7A-7B. The Randles-Sevcik equation predicts that the Faradaic current has a square root dependency on scan rate for diffusion-limited redox reactions. A square root profile is observed in [Fe(CN)_{6]-3/-4}, confirming the reaction is diffusion-limited, as shown in FIGS. 7A-7B. The dependency of the signal increase on scan rate for the transferred electrode is observed in FIG. 7C, in which the normalized ratio is calculated as the ratio at a given scan rate divided by the ratio at the lowest scan rate, 10 mV s⁻¹. A significant change in the signal enhancement with scan rate is evident for the diffusion-limited reaction in [Fe(CN)_{6]3-/4-} than for H₂SO₄, demonstrating that a higher signal enhancement can be achieved at greater scan rates (FIG. 7C).

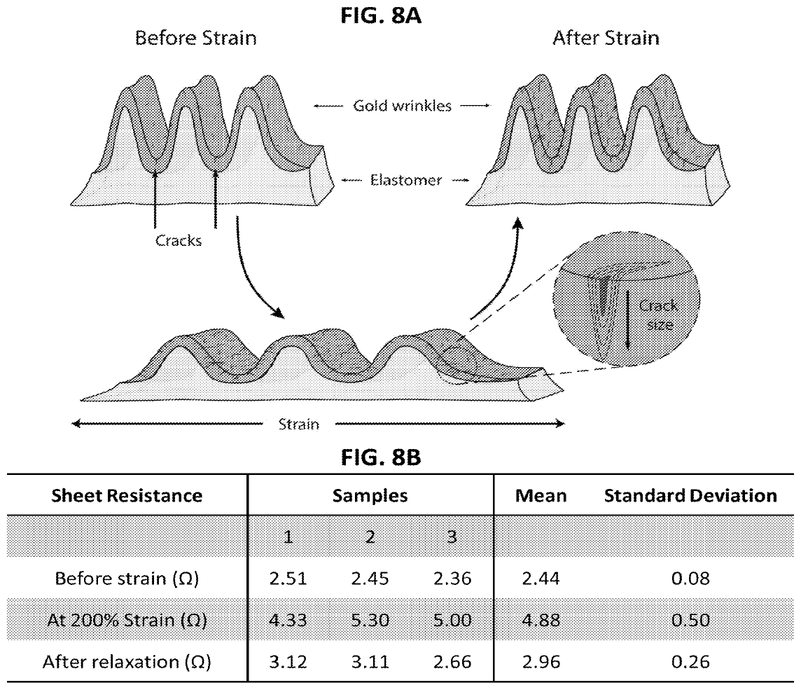

An exemplary embodiment of the present invention is tested by calculating the sheet resistance of transferred electrodes before and after strain, as follows. A Signatone Pro4 Resistivity Test System with an SP4 inline probe is used to measure the sheet resistance of transferred electrodes before, during and after strain. The sheet resistances are first measured on the unstrained electrodes. The same electrodes are stretched up to 200% of the length and the sheet resistance is measured again. The preconditioning step is applied to the electrode, which consisted of straining the sensors up to 200% 10 times. After 5 minutes of relaxation, the sheet resistance of the electrodes is once again measured. FIG. 8B summarizes the sheet resistance values obtained before, during, and after stretching the electrodes up to 200% of the length. The sheet resistance of the wrinkled gold strained to 200% of the original length of the electrode is, on average, approximately double that of the unstrained film. The sheet resistance measured after 5 minutes of relaxation following 10 strain cycles (also to 200% of the original length of the electrode) is, on average, 21% greater than the original, unstrained film. The increase in the resistance is explained by the formation of cracks on the metal film, as illustrated in the simplified schematic (FIG. 8A). Before the electrode is strained, cracks are continuously introduced to the metal film (from handling and natural stress release) and the electrode is mechanically dynamic. Straining the electrodes introduces more cracks to the metal film and increases crack sizes, which results in an increase of the sheet resistance (FIG. 8B). By driving crack formation, the metal film and electron pathways are stabilized, which also likely reduces the noise during electrochemical measurement of surface activity.

An exemplary embodiment of the present invention features glucose detection with a stretchable electrode, as follows. The glucose oxidation potential was measured in a 5×10⁻³ M solution of glucose in PBS (pH 7) by cyclic voltammetry. Chronoamperometry is performed at 0.3 V and glucose was titrated into 20 mL of PBS (pH 7.4) every 30 s. The titration is performed for non-strained electrodes (n=3) and pre-strained electrodes (n=3). An analytical calibration curve is plotted using current values corresponding to sequential additions of glucose. The sensitivity is obtained from the slope and the limit of detection was calculated according to IUPAC

In some embodiments, the present invention features a method for a three-electrode electrochemical mini-cell fabrication. In some embodiments, the method comprises sputtering gold onto polystyrene using a sputtering machine (e.g., QuorumDesign Q150R Plus) and shrinking the gold-sputtered polystyrene in a conventional oven at 130 C. In some embodiments, the method comprises securing the shrunken gold sputtered polystyrene on a laser cutter (e.g., Universal Laser System Ultra R5000) stage and adjusting the power setting to remove all gold from the shrunken gold-sputtered polystyrene sample surface except that which will form the final electrode design. In other embodiments, the method comprises removing the shrunken gold-sputtered polystyrene sample for the laser cutter stage and sonicating in a bath of isopropanol for 5 minutes to remove residues from the surface of the electrodes. In some embodiments, the method results in a shrunken polystyrene substrate with wrinkled gold electrodes in the desired formation. In some embodiments, the three-electrode electrochemical mini-cell is finished when the Ag/AgCl ink is applied to create the third and final electrode.

The present method features a method for producing wrinkled electrodes (200). In some embodiments, the method comprises depositing a metallic film (40) on top of a polymer layer (20) and shrinking the polymer layer (20) and the metallic film (40) to create a shrunken polymer layer (120) and a shrunken metallic film (140). The method further comprises adding Ag/AgCl ink to the electrode. The method further comprises mounting the electrode to an elastomer. In some embodiments the electrodes are functionalized.

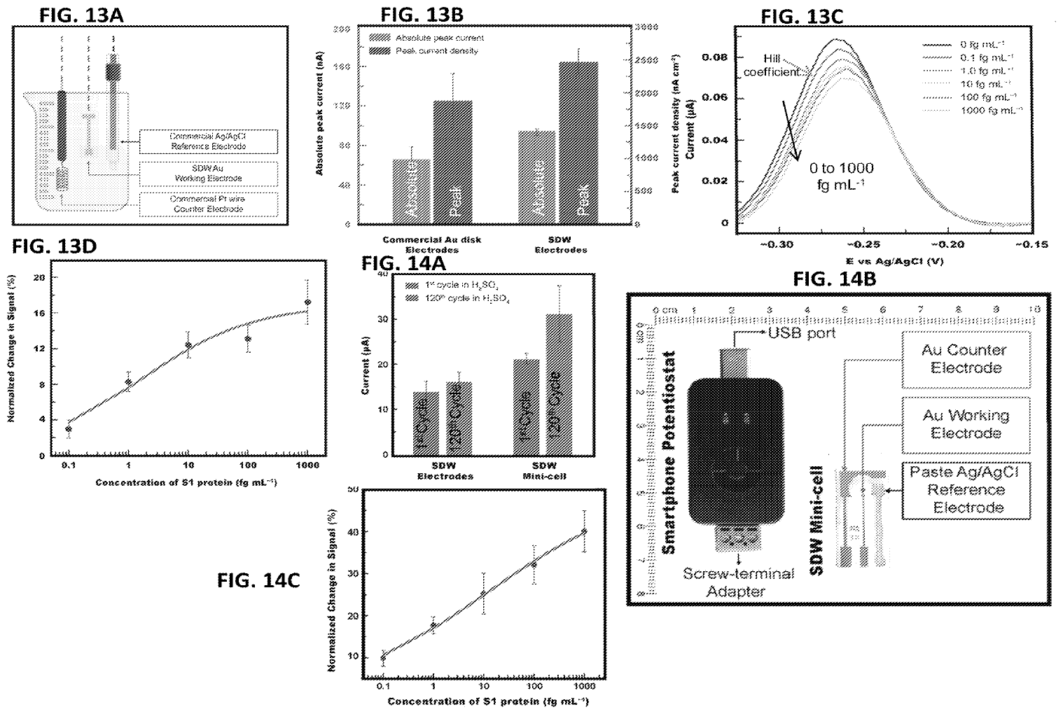

As used herein, a “mini cell” may refer to an electrode that has all three critical components of a 3-electrode electrochemical cell (see FIG. 13A). In some embodiments, the mini cell comprises are at least one reference electrode (Ag/AgCl), at least one counter electrode (i.e., wrinkled Au) and at least one working electrode (i.e., wrinkled Au) (see FIG. 14B). In some embodiments, the working electrode is functionalized.

In some embodiments, the mini cell comprises one or more working electrodes sequentially measured against a reference electrode and a counter electrode. In other embodiments, multiple mini cells each comprising at least one reference electrode, at least one counter electrode and at least one working electrode may be put together to form an array.

In some embodiments, the counter electrode and/or working electrode may be comprised of a wrinkled gold (Au) electrode, or a wrinkled platinum (Pt) electrode, or an electrode made from other suitable materials. In some embodiments, the reference electrode may comprise silver/silver chloride (Ag/AgCl).

As used herein, a “reference electrode” refers to an electrode which has a stable and well-known electrode potential. As used herein, a “counter electrode” refers to an electrode used in two or three electrode systems to complete the circuit so that electrochemical measurements can be performed. As used herein, a “working electrode” refers to the electrode in an electrochemical system on which the reaction of interest is occurring.

In some embodiments, the mini cell may comprise one working electrode. In some embodiments, the mini cell may comprise working two electrodes. In some embodiments, the mini cell may comprise three working electrodes. In some embodiments, the mini cell may comprise four working electrodes. In some embodiments, the mini cell may comprise five working electrodes. In some embodiments, the mini cell may comprise six working electrodes. In some embodiments, the mini cell may comprise seven electrodes. In some embodiments, the mini cell may comprise eight working electrodes. In some embodiments, the mini cell may comprise nine working electrodes. In some embodiments, the mini cell may comprise working ten electrodes. In some embodiments, the mini cell may comprise greater than ten working electrodes.

In some embodiments, the resulting electrode may be flexible. In other embodiments, the resulting electrode may be non-flexible. In some embodiments, the resulting electrode may be functionalized with materials. Non limiting examples of functionalized materials may include but are not limited to aptamers, antibodies, fragments of antibodies, oligonucleotides, or hydrogels containing functionalized material (such as enzymes). In other embodiments, the resulting electrodes may not be functionalized with materials.

Without wishing to limit the present invention to any theory or mechanisms it is thought that stretching the wrinkled surface reveals “hidden” surface area or wrinkled pockets.

The present invention may also feature a method of detecting severe acute respiratory syndrome coronavirus 2 (SARS-CoV-2). In some embodiments, the method comprises obtaining a biological sample from a subject and placing the biological sample on a mini cell. In some embodiments, the mini cell comprises at least one reference electrode (Ag/AgCl), at least one counter electrode (i.e., wrinkled Au) and at least one working electrode (i.e., wrinkled Au). In some embodiments, the working electrode is functionalized with methylene blue (MB)-modified aptamers to detect SARS-CoV-2. In some embodiments, the method comprises detecting square wave voltammograms. In some embodiments, the detection of SARS-CoV-2 causes a decrease in the square wave voltammogram as compared to a reference sample with no SARS-CoV-2.

In some embodiments, the mini cell is connected via electrical leads to the potentiostat, which is connected to a computer which allows for the detection of the square wave voltammograms. In some embodiments, the biological sample or reference sample is incubated on the mini cell for about 1 hour. In some embodiments, the biological sample or reference sample is incubated on the mini cell for about 30 minutes. In some embodiments, the biological sample or reference sample is incubated on the mini cell for about 10 minutes. In some embodiments, the biological sample or reference sample is incubated on the mini cell for over 1 hour.

### EXAMPLES

The following is a non-limiting example of the present invention. It is to be understood that said example is not intended to limit the present invention in any way. Equivalents or substitutes are within the scope of the present invention.

### Example 1

Fabrication and transfer of wrinkled electrodes: Detailed protocol is described below. The average geometric area of the shrunken and transferred electrode was 0.48 and 0.31 cm², respectively. Samples were prepared for SEM imaging with sputtered iridium (6 nm) and were taken using the GAIA Tescan system (8 kV, 0.20 pA).

Fabrication of wrinkled electrodes: PO is mounted onto a silicon wafer to be coated with poly(methyl methacrylate) (PMMA) (Sigma Aldrich) dissolved in toluene (22.5 mg mL-1) to comprise the sacrificial layer. The surface tension of the solvent in which the PMMA was dissolved was taken into consideration to achieve good wetting with the surface energy of the PO, which is in the range of 30 dynes cm2. The surface energy for polyolefins is relatively low, and favors wetting by the lower-surface energy solution. Toluene (28.40 dyne cm-1 at 20° C.) has a lower surface tension than the previously used solvent, anisole (35.00 dyne cm-1 at 20° C.). The PMMA was spin coated at 3200 rpm to ensure uniform spreading and reduce the thickness of the sacrificial layer and subsequently the time necessary to dissolve it. After drying the PMMA film overnight, masks for the electrode design were laser-cut from adhesive and applied to the films as stencils for gold deposition. The initial working area of the electrode was 10.5 cm². An 80 nm gold film was deposited on top of the PMMA-coated PO via sputtering (Quorum 150R). The PO-PMMA-Au is then removed from the silicon wafer substrate and prepared for thermal treatment. The films were shrunk in a convection oven at 140° C. for 13 min. The average geometrical area of the shrunk electrode was 0.48 cm2.

Transfer of wrinkled electrodes to elastomer: The wrinkled gold film was treated with a silane solution to promote bonding to the flexible silicon-based elastomer prior to the liftoff procedure. The elastomer was drop cast onto the silane-treated gold film and allowed to cure overnight at 60° C. The wrinkled films were transferred to the Ecoflex by dissolving the PMMA between the gold and polyolefin in acetone. Once the films were fully transferred, they were placed in an isopropanol bath for an hour and dried overnight. The average geometrical area of the transferred, working electrode was 0.31 cm2. SEM images of samples prepared with sputtered iridium (4 nm) were taken using the GAIA Tescan system (5 kV, 0.20 pA).

Electrochemical characterization: Electrochemical characterization was performed on a Reference 600 potentiostat (Gamry Instruments) with the unshrunk, shrunk, and transferred electrodes as working electrodes, Pt wire as the counter electrode, and Ag/AgCl as the reference electrode. Cyclic voltammetry was firstly performed in a H2SO4 0.5 _{M} solution from 0 to 1.5 Vat 10, 25, 50, 100, 250, 500 and 1000 mV s-1. The process was repeated in 0.005 _{M} [Fe(CN)6]3-/4- solution in 0.05 M PBS solution at pH 7.4 from −0.2 to 0.7 V.

Electrochemical characterization of transferred electrodes under strain: A stretching system was developed to maintain the flexible electrodes under strain while an electrochemical measurement was performed. Cyclic voltammetry was performed at every 30% strain of electrode length in H2SO4 and [Fe(CN)6]3-/4- solutions until it reached 210% of its original length. The electrode was allowed to relax overnight before repeating the stretching procedure.

Glucose detection with stretchable electrode: The glucose oxidation potential was measured in a 5×10-3 _{M} solution of glucose in PBS (pH 7) by cyclic voltammetry. Chronoamperometry was performed at 0.3 V and glucose was titrated into 20 mL of PBS (pH 7.4) every 30 s. The titration was performed for non-strained electrodes (n=3) and pre-strained electrodes (n=3). An analytical calibration curve was plotted using current values corresponding to sequential additions of glucose. The sensitivity was obtained from the slope and the limit of detection was calculated according to IUPAC.

The present invention is directed to stretchable wrinkled electrodes for electrochemical sensing by depositing thin gold metal thin film on polyolefin (PO), which shrinks to 5% of its original area. During the shrinking process, the stiffness mismatch between the polymer and the gold thin film leads to buckling of the gold and results in hierarchical, wrinkled structures. The shrinking factor (the ratio of the average unshrunk electrode's geometric area to that of the processed electrode) of the pre-stressed thermoplastic polyolefin (PO) is 21:8. The wrinkled thin film is then transferred to an elastomer. The transfer of the electrode from shape-memory polymer to elastomer results in additional shrinking due to the lift-off process, with a total shrinking factor of 33:4 (FIG. 2A). The transferred electrodes retain the wrinkled structures (FIG. 2A and FIGS. 2D-2E) and upon application of strain, the wrinkles stretch and separate from each other as cracks are sustained in the gold thin film (FIG. 2C, and FIGS. 2F-2G).

Electrochemical properties of the wrinkled electrodes were characterized by measuring the electrochemical active surface area (EASA) in different solutions. The wrinkled structures contribute to the high surface area, which directly correlates with the Faradaic current. The electrodes were measured by cyclic voltammetry in H₂SO₄ and [Fe(CN)_{6]3-/4-} solutions (FIGS. 6A-6B) and the current densities were obtained by dividing the current by the geometric area (FIGS. 4A-4B). The current densities of shrunk and transferred electrodes were observed to be greater than the unshrunk, planar electrode in both solutions. In some prior systems, a 6.6-fold increase in electrochemical signal was observed in H₂SO₄ for an electrode that had a 20-fold reduction in size, indicating that only a portion of the theoretical surface area was electrochemically active. Even with the application of a hydrophilic polymer to the electrode surface, the EASA increased only by 2-fold, therefore not fully achieving access to the original surface area available before shrinking. Better surface wettability was achieved in this work by modifying the wrinkled surface with oxygen plasma. This method renders the surface hydrophilic and improves surface wetting, removing the need for the hydrophilic polymer coating.

In H₂SO₄, signal enhancements (the ratio of current densities of processed electrodes to unshrunk electrodes) of 21- and 32-fold were achieved for shrunk and transferred electrodes, respectively (FIG. 4C), an improvement of 2.5-fold over prior works. The diffusion independent reaction occurring in the H₂SO₄ solution is the oxidation of gold to gold oxide starting at 1.0 V and the reduction of gold oxide to gold at 0.7 V. The surface area of the shrunk and transferred Au electrodes were calculated by the charge corresponding to the reduction of gold oxide (FIG. 6C). The calculated surface areas match well with the initial geometric area of unshrunk electrodes (10.5 cm²). Therefore, the signal enhancements of 21- and 32-fold in H₂SO₄ match the shrinking factor, suggesting that access to the original surface area was preserved.

Interestingly, the signal enhancements are different depending on the solution in which the measurement was performed. The signal enhancements in the [Fe(CN)_{6]3-/4-} solution are approximately 14- and 13-fold for shrunk and transferred electrodes, respectively. The lower signal enhancement in this solution is due to the [Fe(CN)_{6]3-/4-} reaction being a diffusion limited process with relatively fast electron transfer kinetics. As the potential is applied, the concentration of [Fe(CN)_{6]3-/4-} at the outermost surface of the electrode approaches zero, and a concentration gradient forms in the solution. Diffusion takes place at the surface of the electrodes as the reaction proceeds; however, the diffusion of electroactive species is impeded by the morphology of the wrinkles. A significant portion of the inner surface of the gold wrinkles is therefore not accessed by the [Fe(CN)_{6]3-/4-} ions because the time for diffusion deep into the wrinkles is not sufficient, leading to the disagreement between signal enhancement and shrinking factor. Similarly, the electrochemical behavior of nanoporous gold in high-surface-area, porous electrodes were shown to be biased against redox couples like [Fe(CN)_{6]3-/4-}. A high dependence of signal enhancement on scan rate was more evident for the diffusion-limited reaction in [Fe(CN)_{6]3-/4-} than for H₂SO₄; higher signal enhancement can be achieved at greater scan rates (FIGS. 7A-7C).

The electrochemical behavior of the transferred, stretchable electrodes with applied strain was characterized by stretching the electrodes in H₂SO₄ and [Fe(CN)_{6]3-/4-} up to 210% of its length in increments of 30% (FIGS. 4D-4E). Without wishing to limit the present invention to any theories or mechanisms it is thought that two separate phenomena are occurring simultaneously upon stretching the electrode: (1) an increase in crack formation that eventually stabilizes at a certain number of stretching cycles; and (2) the cracking leads to an increase in EASA. Both phenomena were confirmed as follows. For (1), the electrodes were cycled outside of solution and an increase in resistance upon cycles was witnessed (FIG. 8B) which eventually plateaus (when the cracks are formed). Prior art detailed this phenomenon for strain sensor applications. For (2), a higher electrochemical current was witnessed after stretching. As shown in the voltammograms in FIGS. 4D-4E, stretching the electrode resulted in a greater peak height for the Faradaic current without a significant increase in the capacitive current. Since the Faradaic current scales with surface area, the increase in the signal can be explained by the newly exposed surface area due to crack formation. A 20% increase in signal was observed in H₂SO₄ (FIG. 4F); however, the enhancement to signal after stretching was greater in the [Fe(CN)_{6]3-/4-} solution than in H₂SO₄ (FIG. 4F). This indicated that crack-based formation of new surfaces was not the only phenomenon contributing to the 60% signal enhancement in the [Fe(CN)_{6]3-/4-} solution. It is hypothesized that stretching the wrinkled surface leads to the exposure of wrinkle “pockets” that were previously isolated from the solution and thus not accessed by [Fe(CN)_{6]3-/4-}. This, combined with the newly exposed areas caused by the formation of cracks, resulted in the additional reactive surface area being accessed by [Fe(CN)_{6]3-/4-} and a subsequent increase in current (FIG. 4F). It was visually evident in SEM images (FIG. 2F and FIG. 2G) that the larger-scale wrinkles deform to accommodate the stretching of the substrate at high strains. This results in a surface that is less rough and the [Fe(CN)_{6]3-/4-} ions are able to more easily access the inner surface. The total enhancement to current density increases to approach the shrinking factor as in the H₂SO₄ solution. The stretched, wrinkled electrode, therefore, behaves more similarly to a planar electrode at high strains and allows the present invention to overcome the diffusion limitation of the [Fe(CN)_{6]3-/4-} reaction described above.

Importantly, comparing the voltammograms of unstrained electrodes before and after stretching, a gain in signal of 16 and 13% in H₂SO₄ and [Fe(CN)_{6]3-/4-} respectively was observed (FIG. 4G-4H). This gain in the signal can be attributed to the cracks formed during the straining protocol. Further evidence of wrinkled deformation facilitating diffusion in [Fe(CN)_{6]3-/4-} is that after initial crack formation and relaxation, stretching the electrode again continues to increase the current in [Fe(CN)_{6]3-/4-} solution. Conversely, in H₂SO₄ the current does not significantly increase with strain upon subsequent stretching of the electrode (FIG. 4I). The increase in current with strain in the [Fe(CN)_{6]3-/4-} solution after relaxation demonstrated that access to the surface area can be controlled by stretching the electrode to facilitate diffusion and increase EASA. Straining the electrode was thus used as a preconditioning step to maximally increase the electrical response of the transferred electrodes, which can further improve the detection of biomarkers at low concentration in sweat.

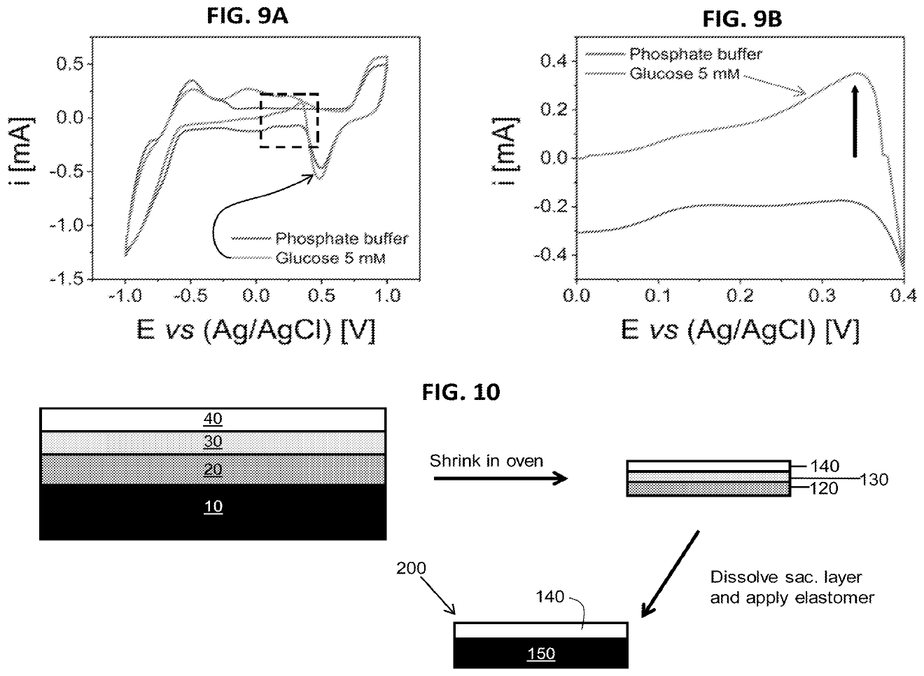

The transferred electrodes were thus evaluated for label- and enzyme-free electrochemical detection of glucose in the clinically relevant concentration range found in sweat, saliva, and tears. Glucose is a commonly studied biomarker, and as a proof of concept, the measurements were taken at physiological pH. The transferred electrode detects glucose without the use of enzymes through its oxidation mechanism at 0.3 V (FIG. 9A-9B).

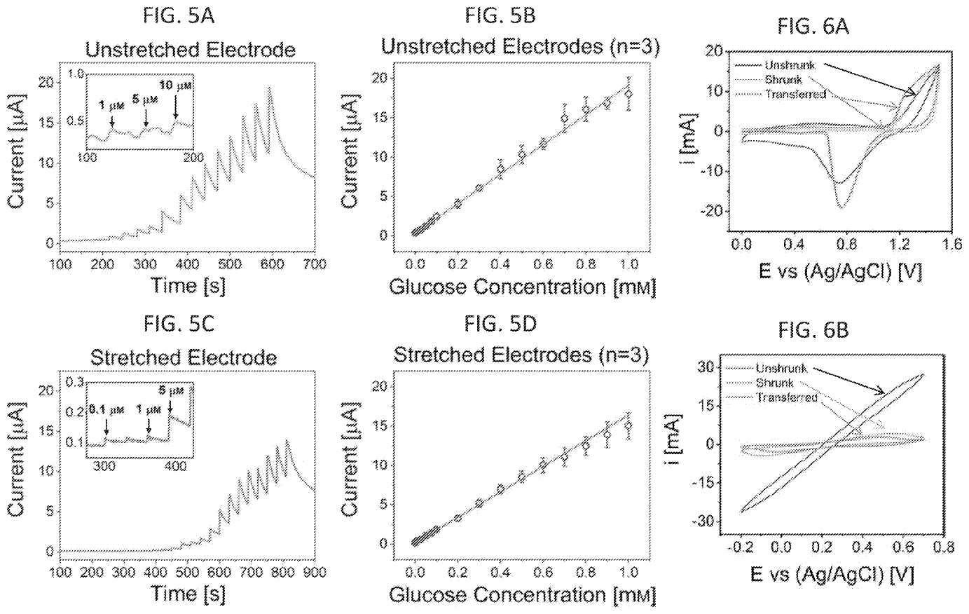

Chronoamperometry was used to measure sequential addition of glucose to PBS pH 7.2 (FIG. 5). The unstrained, transferred electrode showed a LOD of 2.68×10⁻⁷ M which is adequate for glucose detection in the sweat of hypoglycemic and hyperglycemic patients (2×10⁻⁵-6×10⁻⁴ _{M}). The detection of glucose using pre-strained electrodes was evaluated after being stretched up to 210% of its length. The pre-strained electrodes exhibited a broader linear range of detection (1×10⁻⁷-1×10⁻³ _{M}) due to a significant improvement to signal to noise ratio (SNR) of an order of magnitude. The corresponding LOD of pre-strained electrodes was 2.22×10_{-8 M}, an order of magnitude lower than in unstrained electrodes. It is hypothesized that the improved LOD is due to an improvement in SNR, attributed to the mechanical stabilization of the wrinkled thin film. Before the strain cycling, cracks are continuously introduced to the metal film (from handling and natural stress release) and the electrode is mechanically dynamic. By driving crack formation (FIG. 8A), the metal film and electron pathways are stabilized, which also likely reduces the noise during electrochemical measurement of surface activity. After straining the electrode outside of the solution, the standard deviation of the noise of resistance measurements decreased by almost 6-fold, on average, while the standard deviation of the noise of an electrode measured before a glucose injection in solution decreased by an order of magnitude after straining.

The achieved LOD is lower than those reported for electrodes utilizing structures with nanoparticles of gold, platinum, or other alloy combinations with carbon, as well as for nanoporous substrates and electrodes functionalized with glucose oxidase. Table 1 (below) compares the figures of merit obtained in the present invention with other published works investigating flexible, glucose sensors. The range of detection achieved using the flexible, pre-strained electrodes is relevant for monitoring blood sugar via sweat in diabetic patients, as well as for noninvasive methods utilizing saliva, tears, or any application that requires detection of small changes in glucose concentration.

TABLE 1

| | | Linear range | Sensitivity | LOD | Use of |
|---|---|---|---|---|---|
| Group | Electrode Material | [M] | [A × M⁻¹ × cm⁻²] | [M] | Enzyme |
| This work | Wrinkled Au | 1 × 10⁻⁷-1 × 10⁻³ | 0.047 | 2.2 × 10⁻⁸ | No |
| Toi et al.^{[7]} | Reduced graphene oxide- | 1 × 10⁻⁶-1 × 10⁻³ | 0.140 | 5 × 10⁻⁷ | No |
| | polyurethane with Au wrinkles | | | | |
| Chan et al.^{[8]} | Wrinkled Au | 1 × 10⁻³-1 × 10⁻² | 0.860 | 9 × 10⁻⁴ | No |
| Sedighi et al.^{[9]} | NiP0.1- | 1 × 10⁻⁶-1 × 10⁻³ | 1.625 | 1.3 × 10⁻⁷ | No |
| | SnOx/PANI/CuO/cotton | 1 × 10⁻³-1 × 10⁻² | 1.325 | | |
| Bae et al.^{[10]} | Nanoporous Au | 1 × 10⁻⁵-1 × 10⁻³ | 0.2534 | nra) | No |
| Yoon et al.^{[11]} | Nanoporous Pt | 0-1.2 × 10⁻² | 6.84 × 10⁻⁶ | 3 × 10⁻⁵ | No |
| | coated with Nafion | | | | |
| Zhai et al.^{[12]} | Nanowire Au functionalized | 0-8 × 10⁻⁴ | 2.37 × 10⁻² | 1 × 10⁻⁵ | Yes |
| | (NaOH) | | | | |
| Zhao et al.^{[13]} | Au Fiber (PBS) | 0-5 × 10⁻⁴ | 1.17 × 10⁻² | 2 × 10⁻⁵ | Yes |
| Bandodkar et al.^{[14]} | Prussian Blue carbon ink | nra) | 2.3 × 10⁻² | 3 × 10⁻⁶ | Yes |
| Lee et al.^{[15]} | Porous Au with Prussian Blue | 1 × 10⁻⁵-1 × 10⁻³ | nra) | nra) | Yes |
| | and Nafion | | | | |
| Xuan et al.^{[16]} | Reduced graphene oxide with | 0-2.4 × 10⁻³ | 4.5 × 10⁻² | 5 × 10⁻⁶ | Yes |
| | Au and Pt alloy nanoparticles | | | | |
| Yang et al.^{[17]} | Cr/Au/PEDOT: PSS | 3 × 10⁻⁸-3 × 10⁻⁴ | nra) | 3 × 10⁻⁸ | Yes |
| Lin et al.^{[18]} | Ni/Cu/MWCNT | 2.5 × 10⁻⁸ -8 × 10⁻⁴ | 2.633 | 2.5 × 10⁻⁸ | No |
| | | 2 × 10⁻³-8 × 10⁻³ | 2.437 | | |
| Liu et al.^{[19]} | In₂O₃ nanoribbons on Au | 1 × 10⁻⁸-1 × 10⁻³ | nra) | 1 × 10⁻⁸ | Yes |
| Pellitero et al.^{[20]} | Graphite modified with | 1 × 10⁻⁴-1 × 10⁻³ | 0.08 | 1 × 10⁻⁶ | Yes |
| | Prussian Blue | | | | |

^{[7]}P. T. Toi, T. Q. Trung, T. M. L. Dang, C. W. Bae, N.-E. Lee, ACS Appl. Mater. Interfaces 2019, DOI 10.1021/acsami.8b20583;
^{[8]}Y. Chan, M. Skreta, H. McPhee, S. Saha, R. Deus, L. Soleymani, Analyst 2018, 144, 172;
^{[9]}A. Sedighi, M. Montazer, S. Mazinani, Biosens. Bioelectron. 2019, 135, 192;
^{[10]}C. W. Bae, P. T. Toi, B. Y. Kim, W. I. Lee, H. B. Lee, A. Hanif, E. H. Lee, N.-E. Lee, ACS Appl. Mater. Interfaces 2019, 11, 14567;
^{[11]}H. Yoon, X. Xuan, S. Jeong, J. Y. Park, Biosens. Bioelectron. 2018, 117, 267;
^{[12]}Q. Zhai, S. Gong, Y. Wang, Q. Lyu, Y. Liu, Y. Ling, J. Wang, George. P. Simon, W. Cheng, ACS Appl. Mater. Interfaces 2019, DOI 10.1021/acsami.8b19383;
^{[13]}Y. Zhao, Q. Zhai, D. Dong, T. An, S. Gong, Q. Shi, W. Cheng, Anal. Chem. 2019, acs. analchem.9b00152.
^{[14]}A. J. Bandodkar, W. Jia, C. Yardimci, X. Wang, J. Ramirez, J. Wang, Anal. Chem. 2015, 87, 394;
^{[15]}H. Lee, C. Song, Y. S. Hong, M. S. Kim, H. R. Cho, T. Kang, K. Shin, S. H. Choi, T. Hyeon, D.-H. Kim, Sci. Adv. 2017, 3, e1601314;
^{[16]}X. Xuan, H. S. Yoon, J. Y. Park, Biosens. Bioelectron. 2018, 109, 75;
^{[17]}A. Yang, Y. Li, C. Yang, Y. Fu, N. Wang, L. Li, F. Yan, Adv. Mater. 2018, 30, 1800051;
^{[18]}K.-C. Lin, Y.-C. Lin, S.-M. Chen, Electrochimica Acta 2013, 96, 164;
^{[19]}Y. Liu, H. Teng, H. Hou, T. You, Biosens. Bioelectron. 2009, 24, 3329;
^{[20]}M. Aller-Pellitero, J. Fremeau, R. Villa, G. Guirado, B. Lakard, J.-Y. Hihn, F. J. del Campo, Sens. Actuators B Chem. 2019, 290, 591.

In summary, the present invention is directed to high surface area electrodes on soft, stretchable substrates for electrochemical, impedimetric, capacitive, and colorimetric detection of nanomolar quantities of glucose at physiological pH. The method for fabricating the enhanced EASA electrodes requires neither a cleanroom, nor specialty materials or equipment. The wrinkled, transferred gold electrode shows a 32-fold increase in current density compared to a planar electrode of equal geometric size. Stretching the electrode introduces cracks to the wrinkled film and provides additional surface area. Stretching the electrode also resulted in the “unfolding” of some of the wrinkles, which facilitated electroactive species to reach areas not accessed otherwise in reactions limited by diffusion, such as in the redox reaction of [Fe(CN)_{6]3-/4-}.

Straining the electrode therefore functions as a preconditioning step to improve the SNR and enhance the LOD by an order of magnitude over unstrained electrodes. These improvements are crucial for overcoming the challenge of detecting low concentrations of biomarkers. The pre-strained, wrinkled gold electrodes demonstrate a LOD of 2.22×10_{-8 M}, among the lowest reported for enzyme-free glucose detection.

In conclusion, the present invention features a stretchable, electrochemical electrode that has a high surface area allowing for 32-fold enhancement of signal. Stretching the electrode as a conditioning treatment improves the quality of the signal through crack formation by mechanically stabilizing the metal thin film and its electrical characteristics, as well as further increasing the electrochemically active surface area. Furthermore, the device described herein allows for the ability to detect biomarkers like glucose at concentrations as low as 20 nM in physiological solutions.

### Example 2

The following is a non-limiting example of the present invention. It is to be understood that said example is not intended to limit the present invention in any way. Equivalents or substitutes are within the scope of the present invention.

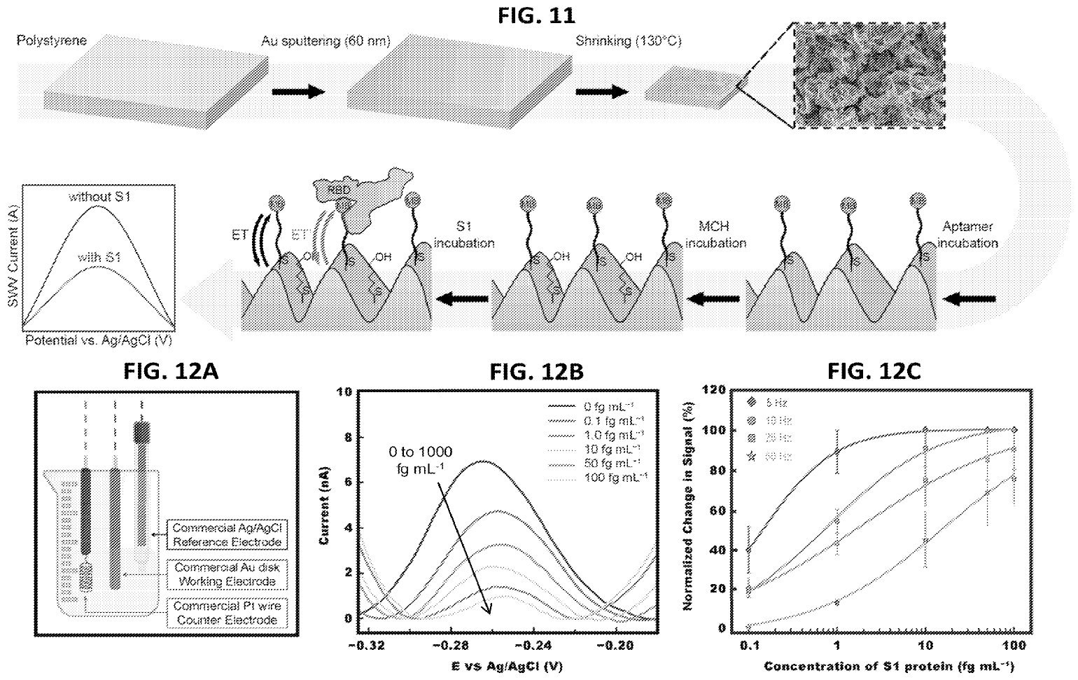

It has been demonstrated that signal enhancement in the form of greater dynamic range of detection on electrodes made using the '80's children's toy, Shrinky-Dink®, which saturates the color of a drawing made on a thermoplastic polystyrene sheet that shrinks when heated (FIG. 11). Here it is demonstrated that using Shrinky-Dink wrinkled (SDW) electrodes for sensitive detection of the S1 subunit of the spike protein of SARS-CoV-2 with the purpose of providing a low-cost screening and diagnostic device for the COVID-19 infection. FIG. 11 demonstrates the fabrication process of SDW electrodes, beginning with sputtering pre-stressed polystyrene with gold, followed by shrinking and immobilization of aptamers and 6-mercaptohexanol (MCH). The structured surface of the SDW gold electrode, shown in the scanning electron microscopy image, supports a high surface area for loading of MB-modified aptamers, which respond to the S1 protein domain by decreasing electron transfer currents from methylene blue (MB).

To determine the functionality and affinity of the aptamers in the aptamer-based electrochemical (E-AB) format, all initial experiments were first performed on commercial disc (CD) Au electrodes (CH Instruments, 2 mm diameter) in a standard 3-electrode electrochemical cell (FIG. 12A). The commercial gold electrodes were functionalized with the aptamers and backfilled with blocking MCH monolayers. Upon exposure of the electrode surface to a buffered solution containing S1 protein, a decrease in the MB peak height of square wave voltammograms was observed (FIG. 12B). The binding of a relatively large protein, such as the S1 protein (78.3 kDa), to the MB-modified aptamer produces an increase in resistance to the transfer of electrons from MB to the electrode (presumably via steric hindrance), leading to a decrease in the MB peak. In the E-AB sensors, the mechanism of detection relies on binding-induced conformational changes that, in turn, alter the electron transfer rate between a redox reporter and the surface of the electrode. The distance between the reporter and the electrode surface directly affects electron transfer kinetics in the system; at small distances, and with little obstruction, electron transfer occurs at a faster rate than at greater distances or in the presence of obstructing molecules. These differences in electron transfer rates translate to differing time constants of current passed, which become relevant when considering measurement parameters. For the SARS-CoV-2 RBD aptamer used in this study, no conformational change has yet been demonstrated. Thus, it is thought that the changes in signal observed in this study upon target binding are due to the physical obstruction of electron transfer from the reporter to the gold surface by bound spike proteins.

The change in peak current correlates with increasing concentrations of S1 protein, as shown in FIG. 12B. The change in signal was evaluated in response to increasing S1 protein concentrations on CD Au electrodes by performing a titration curve in phosphate-buffered solution (FIG. 12C). Measurements were taken at square-wave frequencies ranging from 5 to 50 Hz. Non-linear regression using the Hill isotherm was used to determine the optimal square-wave frequency producing maximum signal gain and broadest dynamic range. This analysis resulted in Hill parameters that reported varying receptor-ligand affinity (KD) and binding stoichiometry with square-wave frequency. The Hill coefficient n was ˜1 at 5 Hz and the data displayed highest sigmoidicity at this frequency, indicating single binding site physics for the interaction between the S1 protein and the aptamer. Frequencies above 5 Hz produced isotherms with slightly lower n values (e.g., n=0.78 at 50 Hz) but broader dynamic range. Based on these calibrations, 10 Hz was chosen as the optimal compromise between sensor affinity and sensitivity for our platform.

The signal change in response to addition of S1 protein on CD electrodes confirmed the viability of the chosen aptamer probes; however, using CD electrodes for screening and diagnostic applications is not feasible. CD electrodes cost $90 each, and given the contagious nature of the disease, reusing them presents significant practical challenges. For a truly deployable and scalable approach, the entire sensor must be disposable. With such considerations of cost of production and scaling in mind, electrodes fabricated with a simple sputtering deposition process on a commercial polystyrene substrate were investigated. Before the shrinking process, the adhesion of sputtered gold on polystyrene is too weak and the gold thin film can delaminate. When shrunk, however, the resulting Shrinky-Dink wrinkled (SDW) electrodes are extremely robust and retain their original surface area, resulting in enhanced current density and dynamic range. Because of the advantages in cost and performance, experiments were initiated on SDW gold electrodes (FIG. 13A) with the goal of detecting the S1 protein directly in saliva.

The total accessible electrochemically active surface area (EASA) of SDW electrodes was confirmed to be greater than that of commercial disc (CD) electrodes, as calculated by integration of the reduction peak of gold oxide in sulfuric acid for the respective electrode types. The electrodes were of equivalent geometric areas. The wrinkles provide a diffusion-based challenge to surface coverage, as previously described. With longer incubation time, however, this limitation was overcome. The outcome was a greater number of moles of aptamers tethered to the surface of SDW electrodes relative to the same geometric area of CD electrodes. The increased aptamer density resulted in greater absolute MB peak heights on SDW electrodes. FIG. 13B demonstrates the MB peak current densities of CD and SDW electrodes are comparable, with the CD electrodes displaying greater variability as measured by the magnitude of the standard deviation between 3 independent electrodes.

To determine whether the introduction of S1 protein to saliva would produce a differentiable signal change relative to untreated saliva, titration curves were built by measuring square wave voltammograms at 10 Hz with SDW electrodes exposed to varying concentrations of S1 protein. The change in signal with increasing concentrations of S1 protein is shown in FIG. 13C. A slight shift in the reduction voltage of MB was observed and attributed to binding-induced changes in the local pH at the surface of the electrode. The titration curve displayed in FIG. 13D was derived from two electrode sets. One set was incubated in saliva, and the second in saliva spiked with S1 protein. Both sets were incubated in saliva for the same total amount of time.

As evidenced from FIG. 13D, SDW electrodes showed a smaller overall signal change than CD electrodes; this is attributed to the SDW electrode measurements being performed in saliva, as opposed to buffer solution as in FIG. 12A-12C. A loss of signal gain in measurements collected from human media compared to buffer solution has been documented previously. The standard error of signal change was lower in SDW electrodes than in CD electrodes, however, which demonstrated the robustness of the fabrication process of SDW electrodes.

To improve the portability of the sensor, next an entire miniaturized electrochemical cell was fabricated, abbreviated as “mini cell”, using the same shrinking process used for individual SDW electrodes. The SDW mini cells' working and counter electrodes were created by sputtering gold onto the pre-stressed polymer substrate and heated to induce shrinking. Ag/AgCl ink was applied as a reference electrode on the shrunk substrate; thus, all three electrodes were located on one substrate, allowing electrochemical measurements within a 250 μL droplet instead of the 20 mL used in the beaker cell. The performance of the SDW mini cell was evaluated by characterizing the gold oxidation and reduction in sulfuric acid. Compared to measurements collected using the commercial reference and counter electrodes, the SDW mini cell demonstrated similar performance, with slightly increased peak-to-peak separation (ΔEp) between oxidation and reduction peaks.

It was observed that during the cleaning protocol performed in sulfuric acid, the area under the peak of the SDW mini cell increased more than that of individual SDW electrodes (FIG. 14A). Without wishing to limit the invention to any theories or mechanisms it was hypothesized that because the SDW mini cell contains an additional source of gold (the counter electrode), there may be a greater rate of gold deposition onto the working electrode occurring during the sulfuric acid cycling, leading to a greater final EASA in the SDW mini cell. The peak height stabilized by the 120th cycle, after which the functionalization of the working electrode on the mini cell proceeded in the same way as for SDW electrodes (FIG. 14B). Next the SDW mini cell was evaluated for detection of the S1 protein in saliva. The SDW mini cell was connected to the PalmSens USB drive-sized potentiostat (FIG. 14B) with alligator clips to demonstrate collection of measurements in any location (non-lab environment).

The signal gain of 40% at the highest concentration of spike protein was greater in the SDW mini cells than in the individual SDW electrodes, in which the maximum change was approximately 16% (FIG. 14C). Because the aptamer functionalization protocol (incubation time, aptamer and MCH concentrations) was the same for both individual SDW electrodes and SDW mini cells, the larger surface area of the SDW mini cell working electrode resulted in a lower probe density. This was also reflected in the differences in peak current densities of MB between individual SDW electrodes and SDW mini cells. Previous studies have shown that a lesser probe density can contribute to greater sensitivity to signal change, which was found to be the case in this study. It was therefore demonstrated that the detection of the 51 protein in the same range tested for individual SDW electrodes, but with greater signal change in the SDW mini cells. From these results, it is believed that the SDW mini cell to be a promising option for further investigation of portable individual tests.

The SDW mini cell demonstrated detection of as little as 0.1 fg mL-1 S1 protein in 10% saliva, with the full comparison to system noise. In comparison, a recent study utilizing the same aptamer to target the RBD region of the spike protein achieved a LOD six orders of magnitude greater in buffer solution than the lowest concentration presented here. Additionally, the low-complexity process does not involve the use of costly materials and does not rely on enzymatic reactions or biotinylation, which is known to cause interference with clinical samples

SDW Electrode Fabrication: The electrode design was created by applying an adhesive polymer (Grafix Frisket Film, Grafix Arts, OH) shadow mask stencil to the polystyrene (Grafix Shrink Film KSF50-C, Grafix Arts, OH) prior to sputtering. A Q150R Plus—Rotary Pumped Coater was used to sputter 60 nm of gold onto the pre-stressed polystyrene to create the working electrode. The substrate was de-masked and placed into a conventional oven at 130° C. for 13 minutes until fully shrunk. Following this step, the electrode was treated with oxygen plasma for 3 minutes to achieve temporary hydrophilicity to ensure full surface wettability during the following cleaning step.

SDW mini cell Fabrication: Electrodes were created by applying an adhesive mask to the polystyrene prior to sputtering. Following the same protocol as for individual SDW electrodes, 60 nm of gold was sputtered onto the pre-stressed polystyrene to create the working and counter electrode. The substrate was then de-masked and placed into a conventional oven at 130° C. for 13 minutes until fully shrunk. Following this step, the reference electrode was painted onto the shrunk polystyrene using commercially available Ag/AgCl ink (Creative Materials). Working and counter electrodes were treated with oxygen plasma for 3 minutes to achieve temporary hydrophilicity, while the Ag/AgCl electrode was covered to prevent oxidation.

Cleaning electrodes: Commercial electrodes were polished using the 0.5 μm-sized Ag particles included in the kit provided with the electrodes prior to cycling in sulfuric acid. The electrodes were then individually immersed in a 0.5 M solution of H₂SO₄ and subjected to 120 cycles at 1000 mV s-1 followed by five cycles at 100 mV s-1 in the potential window of 0-1.5 V. Individual SDW electrodes were similarly immersed in a 0.5 M solution of H2SO4 and subjected to 120 cycles at 1000 mV s-1 followed by five cycles at 100 mV s-1 in the potential window of 0-1.5 V. SDW mini cell electrodes were cleaned by drop-casting a 250 μL drop of 0.5 M solution of H2SO4 onto the surface, ensuring coverage of all three electrodes' working areas, and subjected to 120 cycles at 1000 mV s-1 followed by five cycles at 100 mV s-1 in the potential window of 0-1.5 V.

Aptamer preparation: Aptamers were received in liquid form and diluted to a concentration of 100 μM. For each experiment, a small volume of aptamer solution (100 μM) was combined with a reducing buffer (Basepair Technologies) at a 1:2 volume ratio for one hour to reduce the 3′ ends of the aptamers. Following the reduction step, the solution was diluted with a phosphate buffer solution to a final concentration of 1 μM. 1 μM is an excessive concentration to use, but additional studies must be carried out to determine optimal concentration of probes for incubation. Optimal probe density must also be investigated in order to confirm optimal sensor sensitivity. Probe density affects the magnitude of signal change and is assumed to be a function not only of initial concentration of applied aptamers, but also of incubation time and temperature.56 In the case of oligonucleotides greater than 24 bases, such as in this investigation, the probe density is greatly affected by incubation time. From the SDW mini cell experiments, it was evident that a lesser probe density contributed to enhanced signal gain.

Functionalization of Electrodes with aptamers: A 30 μL of the aptamer solution at 1 μM was applied to each electrode and allowed to incubate 4 and 8 hours, respectively, for CD and SDW electrodes, at room temperature. The aptamer incubation time was optimized for SDW electrodes; from previous investigations, it is clear that the wrinkled morphology presents diffusion limitations for molecular access to the surface. Thus, the incubation time of aptamers was increased to ensure adequate surface coverage by the aptamer probes. During this time, the aptamers formed thiol bonds with the gold surface. The presence of the characteristic MB peak at −0.28 V confirmed the attachment of the aptamer to the gold surface. The peak height collected from each type of electrode (CD, SDW individual and mini cell) was normalized to their respective EASA for comparison of current density. The current density was assumed to correlate with the density of MB and the aptamer probes to which it is attached on the electrode surface. The results of a two-sample t-test show a rejection of the null hypothesis at the 5% significance level that the current densities of CD electrodes and individual SDW electrodes have an equal mean, indicating that the individual SDW electrodes may have a greater probe density. We believe this may be attributed to the longer incubation time chosen for SDW electrodes to compensate for diffusion limitations posed by wrinkles. In contrast, due to the greater EASA of the SDW mini cell working electrodes, the probe density was found to be lesser than that of individual SDW electrodes.

Blocking of electrodes with MCH: Blocking the electrode surface avoids nonspecific binding with interfering species in real samples, enhances the stability of aptamers, and passivates any remaining EASA. A six-carbon blocking molecule, MCH, was chosen as the blocking agent because it has been shown to create more stable monolayers than a 2- or 3-carbon molecule, although at the expense of greater conductivity of the SAM achieved using shorter chains. After the incubation with the aptamers, the electrodes were rinsed and incubated with 30 μL of 3 mM MCH in phosphate buffer for 18 and 39 hours, respectively, for CD and SDW electrodes (both individual and mini cell), at 4° C.

Example 3: Using a saliva sample to detect the 51 protein of severe acute respiratory syndrome coronavirus 2 (SARS-CoV-2). The following example describes a detection strategy for the 51 protein of the SARS-CoV-2 using a saliva sample

A 35-year-old man wakes up one morning with a fever of 102.4° F. and tightness in his chest. He is an essential hospital worker, and so believes he may have contracted COVID-19. Therefore, he calls his primary care physician to determine his next steps. The primary care physician decides that he should be brought in for testing since he is at a higher risk for complications because the patient has severe asthma. The man provides a saliva sample and it is placed on a mini cell. After about an hour incubating the mini cell detects the presence of the 51 protein. The man is told to quarantine for two weeks before returning to work.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the below claims are solely for ease of examination of this patent application, are exemplary, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method for producing stretchable wrinkled electrodes (200) for electrochemical sensing, the method comprising:
a) mounting a polymer layer (20) onto a conductive substrate (10);
b) coating the polymer layer (20) with a sacrificial layer (30);
c) applying an electrode shape template (60) on top of the sacrificial layer (30);
d) depositing a metallic film (40) on top of the sacrificial layer (30) and the electrode shape template (60),
e) removing the electrode shape template (60) from the sacrificial layer (30);
f) removing the polymer layer (20), sacrificial layer (30), and metallic film (40) from the conductive substrate (10);
g) shrinking the polymer layer (20), sacrificial laver (30), and metallic film (40) to produce a shrunken polymer layer (120), a shrunken sacrificial layer (130), and a shrunken metallic film (140);
h) treating the shrunken metallic film (140) with a first solution, wherein the first solution promotes bonding between the shrunken metallic film (140) and an elastomer (150); and
i) dissolving the shrunken sacrificial layer (130) between the shrunken metallic film (140) and the shrunken polymer layer (120) using a second solution, thereby producing the stretchable wrinkled electrode (200) comprising a shrunken metallic film (140) and the elastomer (150).

2. The method of claim 1, wherein the polymer layer (20) and the shrunken polymer layer (120) comprise polyolefin.

3. The method of claim 1, wherein the sacrificial layer (30) and the shrunken sacrificial layer (130) comprise poly(methyl methacrylate) (PMMA) dissolved in toluene.

4. (canceled)

5. The method of claim 1, wherein the metallic film (40) and the shrunken metallic film (140) comprise gold.

6. (canceled)

7. The method of claim 1, wherein the first solution comprises silane.

8. The method of claim 1, wherein the second solution comprises acetone.

9. The method of claim 1, wherein the chemical bath comprises isopropanol.

10. The method of claim 1, wherein the elastomer (150) is silicon-based.

11. The method of claim 1, wherein the stretchable wrinkled electrode (200) has an average geometrical area of 0.2 cm to 0.4 cm.

12. (canceled)

13. A stretchable wrinkled electrode (200) whose surface can be modified for selective sensing of specific analytes using electrochemical, impedimetric, capacitive, and colorimetric detection methods, the electrode comprising:
a) an elastomer (150); and
b) a shrunken metallic film (140);
wherein the shrunken metallic film (140) is fabricated by depositing a metallic film (40) on top of a polymer layer (20) by a sacrificial layer (30), placing the polymer layer (20), the sacrificial layer (30), and the metallic film (40) in an oven such that a shrunken polymer layer (120), a shrunken sacrificial layer (130), and the shrunken metallic film (140) are created, and dissolving the shrunken sacrificial layer (130) to detach the shrunken polymer layer (120);
wherein the shrunken metallic film (140) has been treated with a first solution, such that the first solution promotes bonding between the shrunken metallic film (140) and the elastomer (150);
wherein the shrunken metallic film (140) attaches to the elastomer (150); and
wherein the electrode (200) is placed in a chemical bath and dried after construction and prior to use.

14. The electrode (200) of claim 13, wherein the polymer layer (20) and the shrunken polymer layer (120) comprise polyolefin.

15. The electrode (200) of claim 13, wherein the metallic film (40) and the shrunken metallic film (140) comprise gold.

16. The electrode (200) of claim 13, wherein the sacrificial layer (30) and the shrunken sacrificial layer (130) comprise poly(methyl methacrylate) (PMMA) dissolved in toluene.

17. The electrode (200) of claim 13, wherein the shrunken sacrificial layer (130) is dissolved using a second solution comprising acetone.

18. The electrode (200) of claim 13, wherein the first solution comprises silane.

19. The electrode (200) of claim 13, wherein the chemical bath comprises acetone.

20. The electrode (200) of claim 13, wherein the elastomer (150) is silicon-based.

21. A method for producing wrinkled electrodes (200), the method comprising:
a) depositing a metallic film (40) on top of a polymer layer (20); and
b) shrinking the polymer layer (20) and the metallic film (40) to create a shrunken polymer layer (120) and a shrunken metallic film (140).

22. The method of claim 21, wherein the method further comprises adding Ag/AgCl ink to the electrode.

23. The method of claim 21, wherein the method further comprises mounting the electrode to an elastomer.

24.-31. (canceled)
